(19) United States
(12) Patent Application Publication
(10) Pub. No.: US 2004/0142040 A1
(43) Pub. Date: Jul. 22, 2004

(54) Formulation and dosage form providing increased bioavailability of hydrophobic drugs

(76) Inventors: Liang C. Dong, Sunnyvale, CA (US); Ruiping Zhao, Sunnyvale, CA (US); Patrick S. L. Wong, Burlingame, CA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/698,894

(22) Filed: Oct. 31, 2003

Related U.S. Application Data
(60) Provisional application No. 60/423,184, filed on Oct. 31, 2002.

(51) Int. Cl. A61K 9/14

(52) U.S. Cl. 424/489

(57) ABSTRACT

The present invention provides a drug formulation and a dosage form. The drug formulation works to increase the bioavailability of hydrophobic drugs delivered to the gastro-intestinal tract (“GI tract”) of a desired subject. The drug formulation of the present invention is formulated as a self-emuslifying nanosuspension, which forms an emulsion in-situ upon introduction to an aqueous environment. The dosage form of the present invention may be formed using various different materials and may be configured to deliver the drug formulation of the present invention to the GI tract of a subject using any desired mechanism. A controlled release dosage form according to the present invention may be designed to deliver the drug formulation of the present invention at a desired rate over a desired period of time. If designed as a controlled release dosage form, the dosage form of the present invention may be an osmotic dosage form.

### BACKGROUND

This application claims the benefit of U.S. Provisional Application No. 60/423,184, filed Oct. 31, 2002.

1. Field of the Invention

The present invention relates to formulations and dosage forms for the controlled delivery of hydrophobic drugs. In particular, the present invention provides self-emulsifying formulations and controlled release dosage forms that enhance the bioavailability of hydrophobic drugs.

2. State of the Art

To ease dosing and improve patient compliance, it is generally preferred to dose a desired drug using an oral dosage form rather than parenteral administration. However, oral delivery of hydrophobic drug substances has proven challenging. In particular, hydrophobic drug substances tend to exhibit poor or inconsistent bioavailability when administered orally. As it is used herein, the term “bioavailability” refers to the amount of drug that reaches general blood circulation from an administered dosage form. Often drug absorption in the gastro-intestinal tract is driven by the concentration gradient of the drug generated across the gastro-intestinal mucosal membrane (“the mucosa” or “the mucosal membrane”), with the drug absorption increasing as the drug concentration gradient increases. Because hydrophobic drugs do not readily dissolve in the aqueous gastro-intestinal environment, the concentration gradient generated by a hydrophobic drug delivered to the gastro-intestinal tract is small, at best, and results in limited absorption of the drug across the mucosal membrane. The limited bioavailability of orally administered, hydrophobic drugs is particularly problematic when it is considered that approximately 10% of currently marketed drugs exhibit poor water solubility. Even more troubling is the fact that approximately 40% of the newly discovered chemical entities that have potential therapeutic value are not pursued as drugs because of their poor solubility in water. It would be an improvement in the art, therefore, to provide a formulation and dosage form that increase the oral bioavailability of hydrophobic drugs.

Self-emulsifying formulations have been used to increase the bioavailability of hydrophobic drugs. A self-emulsifying formulation generally includes an oil phase, a surfactant, and a drug material. Upon, exposure to an aqueous environment, the oil phase and surfactant, interact to form an emulsion wherein the hydrophobic drug exhibits an increased solubility. A self-emulsifying formulation, therefore, has the potential to increase the solubility of a hydrophobic drug in an aqueous environment, and thereby increase the bioavailability of a hydrophobic drug delivered to the GI tract of a subject. U.S. Pat. Nos. 6,436,430, 6,284,268, 6,221,391, 6,174,547, 6,057,289, 5,965,160, and 5,578,642 discuss various self-emulsifying formulations developed to facilitate oral administration of hydrophobic drugs. It would be desirable to provide a self-emulsifying formulation suitable for oral administration of hydrophobic drugs that increases the solubility of hydrophobic drugs in an aqueous environment such that therapeutic doses of hydrophobic drugs could be orally administered using fewer numbers of dosage forms or a dosage form of a readily acceptable size. Ideally, such a formulation would provide desirable drug loading characteristics, would be compatible with various different dosage forms, would work to reduce aggregation of hydrophobic drug contained within the formulation before delivery to an aqueous environment, and would provide an emulsion that worked to solubilize the hydrophobic drug even for extended periods after delivery of the formulation to an aqueous environment.

### SUMMARY OF THE INVENTION

The present invention provides a drug formulation that works to increase the bioavailability of hydrophobic drugs delivered to the gastro-intestinal tract (“GI tract”) of a desired subject. The drug formulation of the present invention is formulated as a self-emuslifying nanosuspension, which forms an emulsion in-situ upon introduction to an aqueous environment. As they are used herein, the term “subject” refers to an animal, including a human, to which a drug is administered, the term “aqueous environment” indicates an environment containing water or water containing fluids, including in vivo media found in animals, such as the aqueous fluid present in the GI tract of an animal, and the terms “aqueous medium” and “aqueous media” refer to water or water containing fluids, including in vivo media found in animals, such as the aqueous fluid present in the GI tract of an animal.

A self-emulsifying nanosuspension according to the present invention includes a saturated fatty acid, one or more surface acting agents, or surfactants, and nanoparticles of hydrophobic drug dispersed within the fatty acid and one or more surfactants. The self-emulsifying nanosuspension of the present invention facilitates increased loading of hydrophobic drug into a given volume of formulation, is stable over time, greatly increases the solubility of hydrophobic drugs in an aqueous environment, and provides a surprising increase in the bioavailability of orally administered hydrophobic drugs. In addition, the self-emulsifying nanosuspension of the present invention forms an emulsion that works to solubilize hydrophobic drug material for extended periods after delivery of the self-emulsifying nanosuspension to an aqueous environment. As they are used herein, the term “solubilize” means to make soluble or more soluble in an aqueous environment, the term “solution” indicates a chemically and physically homogenous mixture of two or more substances, and the term “solubility” refers to the quantity of a particular substance that can dissolve in a particular solvent.

The present invention also includes a dosage form designed to deliver the self-emulsifying nanosuspension of the present invention. The dosage form of the present invention may be formed using various different materials and may be configured to deliver the drug formulation of the present invention to the GI tract of a subject using any desired mechanism. For example, the dosage form of the present invention may be designed to delay the release of drug formulation for a desired period of time post administration, or the dosage form may be designed to release drug formulation only when exposed to chosen environmental conditions. Additionally, the dosage form of the present invention may be designed to provide the controlled release of drug formulation over a desired period of time or under chosen environmental conditions. A controlled release dosage form according to the present invention may be designed to deliver the drug formulation of the present invention at a desired rate over a desired period of time. If designed as a controlled release dosage form, the dosage form of the present invention may be an osmotic dosage form. In one aspect, the present invention includes an osmotic, controlled release dosage form designed to delay release of drug formulation until after the dosage form has passed through the upper portion of the GI tract of a subject such that substantially all of the formulation is delivered at a controlled rate in the lower GI tract.

### BRIEF DESCRIPTION OF THE DRAWINGS

FIGS. 1 through 8 provide various schematic illustrations of exemplary controlled release soft-cap dosage forms according to the present invention.

FIGS. 9A through 9D provide a series of schematic representations illustrating a method for forming a plug to seal exposed portions of osmotic composition at an exit orifice included in a dosage form according to the present invention.

FIG. 12 through FIG. 14 provide schematic representations illustrating a method of forming a seal on the inner surface of an exit orifice included in a dosage form according to the present invention.

FIG. 15. provides a schematic illustration of an exemplary hard-cap controlled release dosage form according to the present invention.

FIG. 16 provides a graph illustrating the results of a study conducted to evaluate the solubility of raw megestrol acetate and nanoparticulate megestrol acetate in AIF the presence of various concentrations of a self-emulsifying carrier useful in the self-emulsifying nanosuspension of the present invention.

FIG. 17 provides a graph illustrating the results of a study conducted to evaluate the stability of megestrol acetate solubilized in an emulsion formed by a self-emulsifying carrier useful in the self-emulsifying nanosuspension of the present invention.

FIG. 18 provides a graph illustrating the release profile of megestrol acetate provided by a dosage form according to the present invention.

FIG. 19 provides a graph illustrating the release profile of megestrol acetate provided by a second dosage form according to the present invention.

FIG. 20. provides a graph and table setting for the results of a PK study conducted to evaluate the bioavailability of megestrol acetate provided by various different dosage forms, including two different dosage forms according to the present invention.

Table 1 provides physical properties of saturated fatty acids ranging from saturated C6 fatty acids to saturated C18 fatty acids.

Table 2 describes the formulations delivered by the different dosage used in the PK study described in Example 5.

Table 3 details the Liquid Chromotography/Mass Spectroscopy conditions used to evaluate the plasma concentration of megestrol acetate as part of the PK study described in Example 5.

### DETAILED DESCRIPTION OF THE INVENTION

The present invention includes a self-emulsifying nanosuspension. As used herein, the term “nanosuspension” indicates a flowable formulation containing an amount of nanoparticles dispersed therein. The self-emulsifying nanosuspension of the present invention includes an oil phase, one or more surfactants, and nanoparticles of a desired hydrophobic drug. The self-emulsifying nanosuspension of the present invention forms an emulsion in-situ upon exposure to aqueous media and works to enhance the solubility of hydrophobic drug in an aqueous environment. In particular, the self-emulsifying nanosuspension of the present invention enhances the solubility of hydrophobic drug delivered to the GI tract of a subject. The self-emulsifying nanosuspension of the present invention also provides a surprising increase in the bioavailability of orally administered hydrophobic drug and facilitates the manufacture of acceptably sized oral dosage forms capable of delivering therapeutic doses of hydrophobic drug to a subject.

The self-emulsifying nanosuspension of the present invention utilizes saturated fatty acid as an oil phase. Saturated fatty acid is used as the oil phase of the self-emulsifying nanosuspension of the present invention because fatty acids provide a relatively stable oil phase and facilitate more complete delivery of the hydrophobic drug included in the self-emulsifying nanosuspension. Saturated fatty acids are hydrophobic components that do not require the action of lipase to be digested. Where a drug formulation includes a lipid as the oil phase, drug dissolved within the lipid may be trapped and left undelivered if the lipid is not degraded by enzymatic activity. This is of particular concern where the formulation is released from a controlled release dosage form, which may release a large percentage of the drug formulation in the lower GI tract where lipase may not exist or exists in reduced concentrations. By utilizing a saturated fatty acid as the oil phase, the self-emulsifying nanosuspension of the present invention reduces the risk that the hydrophobic drug loaded into the self-emulsifying nanosuspension will be trapped within an undigested oil phase and rendered undeliverable. Moreover, because the fatty acid used in the self-emulsifying nanosuspension is a saturated fatty acid, the self-emulsifying nanosuspension of the present invention reduces the stability issues associated with drug formulations including an unsaturated hydrophilic material, such as an unsaturated lipid or fatty acid. The one or more carbon-carbon double bonds found in unsaturated hydrophilic materials are significantly less stable than the carbon-carbon single bonds and, over time, such instability works to degrade drug formulations that incorporate unsaturated hydrophilic materials.

In order to achieve a self-emulsifying nanosuspension that is flowable at physiologic temperatures, however, the saturated fatty included as the oil phase of the self-emulsifying nanosuspension of the present invention must be chosen carefully. It has been found that saturated fatty acids that are smaller than C8 fatty acids do not exhibit sufficient hydrophobicity to consistently create a multiphase emulsion in-situ upon exposure to aqueous media. Therefore, the self-emulsifying nanosuspension of the present invention is formulated using a saturated fatty acid that is a C8 fatty acid or larger. However, the melting point of saturated fatty acids increases undesirably as the size of the saturated fatty acid increases beyond C12 fatty acids. Even after mixture with one or more excipients, the melting points of saturated fatty acids larger than C12 are too high to provide a flowable drug formulation at physiologic temperatures. Therefore, the oil phase of the self-emulsifying nanosuspension of the present invention is preferably formed using saturated C8 to Cl2 fatty acids. Table 1 provides physical properties of saturated fatty acids ranging from saturated C6 fatty acids to saturated C18 fatty acids.

Though the oil phase of the self-emulsifying nanosuspension of the present invention may include a single type of saturated fatty acid or a mixture of different saturated fatty acids, in each embodiment, the oil phase of the self-emulsifying nanosuspension of the present invention will include an amount of C8, C10, or C12 fatty acid. In a particularly preferred embodiment, capric acid, a saturated C10 fatty acid, serves as the oil phase of the self-emulsifying formulation of the present invention. As can be appreciated by reference to Table 1, capric acid has a melting temperature of 31° C. and a low solubility in water. The self-emulsifying nanosuspension of the present invention includes between about 10 wt % and about 80 wt % saturated fatty acid, with the saturated fatty acid preferrably accounting for about 35 wt % to about 45 wt % of the self-emulsifying nanosuspension.

A variety of different surfactants may be used in the self-emulsifying nanosuspension of the present invention. The one or more surfactants included in the self-emulsifying nanosuspension of the present invention work to reduce the interfacial tension between the hydrophobic components of the nanosuspension and any aqueous media included in the environment into which the nanosuspension is delivered. Thus, upon delivery of the self-emulsifying nanosuspension of the present invention to an aqueous environment, the one or more surfactants included in the formulation work to automatically create a stable emulsion in-situ. The one or more surfactants included in the formulation of the present invention are preferably one or more non-ionic surfactants. For example, surfactants that may be used in the self-emulsifying formulation of the present invention include polyoxyethylene products of hydrogenated vegetable oils, polyethoxylated castor oils or polyethoxylated hydrogenated castor oil, polyoxyehtylene-sorbitan-fatty acid esters, polyoxyethylene castor oil derivatives and the like. The one or more surfactants included in the self-emulsifying nanosuspension of the present invention may include a surfactant selected from polyoxyethylenated castor oil comprising 9 moles of ethylene oxide, polyoxyethylenated castor oil comprising 15 moles of ethylene oxide, polyoxyethylenated castor oil comprising 25 moles of ethylene oxide, polyoxyethylenated castor oil comprising 35 moles of ethylene oxide, polyoxyethylene castor oil comprising 40 moles of ethylene oxide, polyoxylenated castor oil comprising 52 moles of ethylene oxide, polyoxyethylenated sorbitan monopalmitate comprising 20 moles of ethylene oxide, polyoxyethylenated sorbitan monostearate comprising 20 moles of ethylene oxide, polyoxyethylenated sorbitan monostearate comprising 4 moles of ethylene oxide, polyoxyethylenated sorbitan tristearate comprising 20 moles of ethylene oxide, polyoxyethylenated sorbitan monostearate comprising 20 moles of ethylene oxide, polyoxyethylenated sorbitan trioleate comprising 20 moles of ethylene oxide, polyoxyethylenated stearic acid comprising 8 moles of ethylene oxide, polyoxyethylene lauryl ether, polyoxyethylenated stearic acid comprising 40 moles of ethylene oxide, polyoxyethylenated stearic acid comprising 50 moles of ethylene oxide, polyoxyethylenated stearyl alcohol comprising 2 moles of ethylene oxide, and polyoxyethylenated oleyl alcohol comprising 2 moles of ethylene oxide. Such surfactants are available from Atlas Chemical Industries, Wilmington, Del.; Drew Chemical Corp., Boonton, N.J.; and GAF Corp., New York, N.Y. Further examples of commercially available surfactants that may be used in the self-emulsifying nanosuspension of the present invention include: NIKKOL HCO-50®, NIKKOL HCO-35®, NIKKOL HCO-40®, NIKKOL HCO-60(D (from Nikko Chemicals Co. Ltd); CREMAPHORE®, CREMAPHORE RH40®, CREMAPHORE RH60®, CREMAPHORE RH410®, CREMAPHORE RH455®, and CREMAPHORE EL® (from BASF); and Tweens, such as TWEEN 20®, TWEEN 21®, TWEEN 40®, TWEEN 60®, TWEEN 80®, and TWEEN 81® (from ICI Chemicals). Addional surfactants that may be used in the self-emulsifying nanosuspension of the present invention include Pluronic surfactants, such as Pluronic F68, F108, and F127.

The amount of surfactant included in the self-emulsifying nanosuspension of the present invention will depend on a variety of factors. Among such factors are the amount and type of fatty acid and drug included in the formulation, the type of surfactant or surfactants used, and the type of emulsion desired as the self-emulsifying formulation is introduced into an aqueous environment. For example, the self-emulsifying formulation of the present invention may include sufficient surfactant to produce a stable emulsion or microemulsion upon contact with an aqueous medium. As it is used herein, the term “microemulsion” indicates a multicomponent system that exhibits a homogenous oil-in-water emulsion with an average oil-droplet size of less than 1 μm in diameter and in which quantities of a drug can be solubilized. Typically, a microemulsion can be recognized and distinguished from ordinary emulsions in that the microemulsion is more stable and usually substantially transparent or opalescent. However, the self-emulsifying formulation of the present invention may also be formulated to produce an emulsion that is coarser than a microemulsion. Generally, the self-emulsifying formulation will include about 5 wt % to about 90 wt % surfactant, with the self-emulsifying nanosuspension of the present invention preferably including about 25 wt % to about 45 wt % surfactant.

The hydrophobic drug included in the self-emulsifying nanosuspension of the present invention is dispersed within the self-emulsifying nanosuspension as a nanoparticulate material. The term “hydrophobic drug” as it is used herein indicates a drug that may be characterized as a Class II drug under the Biopharmaceutics Classification System with a dose/solubility volume of more than 250 ml. Drugs that may be used in the self-emulsifying nanosuspension of the present invention include, but are not limited to, hydrophobic drugs which are antibacterial agents, antiviral agents, anti-fungal agents, antacids, anti-inflammatory substances, coronary vasodilators, cerebral vasodilators, psychotropics, antineoplastics, stimulants, antihistamines, laxatives, decongestants, vitamins, anti-diarrheal preparations, anti-anginal agents, vasodilators, anti-arrythmics, anti-hypertensives, vasoconstrictors, anti-migraine drugs, antineoplastic drugs, anticoagulants, anti-thrombotic drugs, analgesics, anti-pyretics, neuromuscular agents, agents acting on the central nervous system, hyperglycemic agents, hypoglycemic agents, thyroid and anti-thyroid preparations, diuretics, anti-spasmodics, uterine relaxants, mineral and nutritional additives, anti-obesity agents, anabolic agents, ani-asthmatics, expectorants, cough suppressants, mucolytics, and anti-uricemic drugs. The hydrophobic drug included in the self-emulsifying nanosuspension of the present invention may also be a pharmacologically active but poorly soluble protein, polypeptide, peptide, proteomimetic, or peptidomimetic material.

The solubility of the hydrophobic drug included in the self-emulsifying nanosuspension of the present invention is greater in the oil phase of the self-emulsifying nanosuspension than in water. Preferably, the hydrophobic drug exhibits a solubility in the oil phase of the self-emulsifying nanosuspension of the present invention that is at least ten times greater than the solubility of the hydrophobic drug in water. More preferably, the hydrophobic drug exhibits a solubility in the oil phase of the self-emulsifying nanosuspension of the present invention that is at least 100 times greater than the solubility of the hydrophobic drug in water, and even more preferably, the hydrophobic drug exhibits a solubility in the oil phase of the self-emulsifying nanosuspension of the present invention that is at least 500 times greater than the solubility of the hydrophobic drug in water.

Although the hydrophobic drug included in the self-emulsifying nanosuspension of the present invention is more soluble in the oil phase of the self-emulsifying nanosuspension than it is in water, the hydrophobic drug need not be completely dissolved within the self-emulsifying nanosuspension before delivery of the self-emulsifying nanosuspension to an environment of operation. Instead, the self-emulsifying nanosuspension of the present invention is preferably prepared as a suspension having an amount of hydrophobic drug dissolved within the saturated fatty acid and surfactant as well as an amount of undissolved hydrophobic drug dispersed within the formulation. In a particularly preferred embodiment, the self-emulsifying nanosuspension of the present invention is formulated such that, before delivery to an environment of operation, the amount of undissolved hydrophobic drug dispersed within the self-emulsifying nanosuspension is greater than the amount of hydrophobic drug dissolved within the self-emulsifying nanosuspension. Once delivered to the GI environment of a subject, the self-emulsifying nanosuspension of the present invention facilitates absorption of the hydrophobic drug that is dissolved within the fatty acid forming the oil phase. Moreover, as the hydrophobic drug dissolved in the oil phase of the emulsion formed by the self-emulsifying nanosuspension of the present invention is absorbed or partitions out of the oil phase, the emulsion formed by the self-emulsifying nanosuspension of the present invention provides continued solubilization of previously undissolved hydrophobic drug material dispersed within the formulation.

In order to create the self-emulsifying nanosuspension of the present invention, the hydrophobic drug used in the self-emulsifying nanosuspension is prepared as a nanoparticulate material. As they are used herein, the terms “nanoparticulate” or “nanoparticle” indicate particles that exhibit a mean particles size that is smaller than 1 μm in all dimensions. Preferably, the particles of hydrophobic drug included in the self-emulsifying nanosuspension of the present invention exhibit a mean particle size smaller than about 0.5 μm in every dimension, and most preferably, the particles of hydrophobic drug included in the self-emulsifying nanosuspension of the present invention exhibit a mean particle size that is smaller than about 0.2 μm in every dimension. Though a vacuum mixer, such as a Ross mixer, is presently preferred for dispersing the nanoparticles of hydrophobic drug within the self-emulsifying nanosuspension of the present invention, the nanoparticles of hydrophobic drug may be dispersed within the formulation using any suitable method that results in a nanosuspension as already defined. Moreover, nanoparticles of a desired hydrophobic drug can be prepared for dispersion within the self-emulsifying nanosuspension of the present invention using any process providing particles within a desired range of sizes. For example, the drug may be processed using a wet-milling or supercritical fluid process, such as an RESS or GAS process. In addition, processes for producing nanoparticles are disclosed in U.S. Pat. Nos. 6,267,989, 5,510,118, 5,494,683, and 5,145,684, the contents of which are incorporated herein by reference.

In order to obtain nanoparticulate material, it is generally necessary to process the material with an agent that will coat the particles as they are processed. If material is not processed in the presence of a coating agent, the particulates formed as the material is processed will rapidly aggregate or agglomerate and nanoparticles will not be achieved. Therefore, the self-emulsifying nanosuspension of the present invention will also include an amount of coating agent used to prevent aggregation or agglomeration of the nanoparticles of hydrophobic drug. Exemplary coating agents include lipids, hydophilic polymers, such as hydroxypropyl methylcellulose (“HPMC”) and polyvinylpyrrolidone (“PVP”) polymers, and solid or liquid surfactants. The coating agent used in a nanoparticle forming process may also include a mixture of agents, such as a mixture of two different surfactants. Where used as a coating agent, a hydrophilic polymer may work to both facilitate formation of nanoparticulate material and stabilize the resulting nanoparticles against recrystalization over long periods of storage. Surfactants useful as coating agents in the creation of nanoparticles useful in the self-emulsifying nanosuspension of the present invention include nonionic surfactants, such as Pluronic F68, F108, or F127. The non-ionic surfactants already mentioned herein may also be useful as coating agents in a nanoparticle forming process.

The amount of coating agent included in the self-emulsifying nanosuspension of the present invention will depend on the amount of hydrophobic drug material dispersed within the suspension. However, the amount of coating agent included in the nanoparticulate, hydrophobic drug material included in the self-emulsifying nanosuspension of the present invention preferably ranges from about 10 wt % to about 70 wt %, with the hydrophobic drug material representing from about 30 wt % to about 90 wt % of the nanoparticulate material. Preferably, the nanoparticulate, hydrophobic drug material included in the self-emulsifying nanosuspension of the present invention includes about 25% to about 35% coating agent and about 65% to about 75% hydrophobic drug material, with the total wt % of coating agent and drug material equaling 100 wt %.

Preparing the self-emulsifying nanosuspension of the present invention with nanoparticles of hydrophobic drug allows increased drug loading. Preparing the hydrophobic drug as a nanoparticulate material facilitates increased drug loading of the self-emulsifying nanosuspension without compromising bioavailability. It has been found that, compared to coarser material, more nanoparticulate drug material can be dispersed within a self-emulsifying formulation without causing segregation of the drug material or otherwise adversely affecting the stability of the self-emulsifying suspension. Moreover, the use of nanoparticulate hydrophobic drug material allows the formulation of a substantially uniform suspension of drug material in a low viscosity self-emuslifying carrier, as nanoparticles of hydrophobic drug do not exhibit settling even when dispersed in a low viscosity liquid. In contrast, where larger particles, even microparticles, are dispersed to form a suspension, a viscosity enhancing agent is necessary to maintain a uniform suspension and prevent settling, and such higher viscosity formulations may not be well suited for delivery from a controlled release delivery device. The increased drug loading permitted by the self-emulsifying nanosuspension of the present invention allows relatively more hydrophobic drug to be delivered from a given volume of drug formulation, which, in-turn, can reduce the size of dosage form required to administer a given dose of a desired hydrophobic drug.

The amount of drug included in the self-emulsifying nanosuspension of the present invention will vary depending on the drug used and the desired dose to be delivered. Generally, self-emulsifying formulation of the present invention will include enough hydrophobic drug material to deliver about 10 mg to about 250 mg of hydrophobic drug from an acceptably sized dosage form. In a preferred embodiment, the self-emulsifying nanosuspension of the present invention includes enough hydrophobic drug material to deliver about 40 mg to about 150 mg of hydrophobic drug from an acceptably sized dosage form. Alternatively, the self-emulsifying nanosuspension of the present invention preferably includes from about 2 wt % to about 50 wt % hydrophobic drug, and in particularly preferred embodiments, the self-emulsifying nanosuspension of the present invention includes from about 10 wt % to about 30 wt % hydrophobic drug

Beyond its drug loading characteristics, the self-emulsifying nanosuspension of the present invention enhances the solubility of hydrophobic drug in an aqueous environment, and the emulsion formed by the self-emulsifying nanosuspension of the present invention works to prevent precipitation of the fraction of hydrophobic drug solubilized within the emulsion. The emulsion formed by the saturated fatty acid and surfactant included in the self-emulsifying nanosuspension of the present invention have been shown to maintain the solubility of hydrophobic drug material for a period of hours after introduction into an aqueous fluid, such as artificial intestinal fluid (“AIF”).

The self-emulsifying nanosuspension of the present invention is also compatible with various dosage forms, which allows the self-emulsifying nanosuspension of the present invention to be easily administered orally. Due to the increased solubility provided by the self-emulsifying nanosuspension of the present invention, the self-emulsifying nanosuspension facilitates the creation of a relatively higher concentration of dissolved hydrophobic drug in the GI tract of a subject. Moreover, because the emulsion formed by the self-emulsifying nanosuspension works to solubilize hydrophobic drug as the dissolved drug material is absorbed, the self-emulsifying nanosuspension of the present invention works to maintain a higher concentration of dissolved hydrophobic drug over a longer period of time than would be possible if the formulation simply included an amount of dissolved hydrophobic drug. Therefore, as it is delivered to the GI tract of a subject using an oral dosage form, the self-emulsifying nanosuspension of the present invention works both to create and maintain a higher concentration of dissolved hydrophobic drug within the GI tract. By working to both create and maintain a higher concentration of dissolved hydrophobic drug within the GI tract of a subject, it is believed that the self-emulsifying nanosuspension of the present invention works to increase transport of hydrophobic drug across the mucosal membrane and thereby works to increase the bioavailabilty of hydrophobic drug administered using an oral dosage form.

The self-emulsifying nanosuspension of the present invention can be administered to a subject using any oral dosage form that is capable of containing the self-emulsifying nanosuspension of the present invention, is compatible with the self-emulsifying nanosuspension, and can deliver the self-emulsifying nanosuspension of the present invention to the GI of the subject. However, it has been found that the self-emulsifying nanosuspension of the present invention provides a surprising increase in bioavailabilty when delivered to the GI tract of a subject using a controlled release dosage form.

It is believed that the combination of at least two factors lead to the relatively higher bioavailability achieved where the self-emulsifying nanosuspension of the present invention delivered using a controlled release dosage form. First, the solubility of the hydrophobic drug in an aqueous environment increases as the concentration of the self-emulsifying nanosuspension in the aqueous environment increases. In particular, it has been found that the even small increases in the concentration of self-emulsifying nanosuspension in an aqueous environment can provide large increases in the solubility of the hydrophobic drug. Second, controlled release dosage forms tend to deliver an amount of the drug formulation contained within the dosage forms to the lower portions of the GI tract of the subject, and the lower portions of the GI generally contain less aqueous media than the upper GI tract. Therefore, a controlled release dosage form works to deliver an amount of the self-emulsifying nanosuspension of the present invention to an environment containing relatively less aqueous media, which is believed to provide a relatively higher concentration of self-emulsifying nanosuspension at the location of delivery. The higher concentration of the self-emulsifying nanosuspension, in turn, is believed to increase the solubility of the hydrophobic drug in the GI environment and thereby enhance the oral bioavailability of the hydrophobic drug.

The present invention includes a controlled release dosage form. A controlled release dosage form according to the present invention includes any controlled release dosage form capable of containing the self-emulsifying nanosuspension of the present invention, is compatible with the self-emulsifying nanosuspension of the present invention, and delivers the self-emulsifying nanosuspension of the present invention at a controlled rate over a desired period of time within the GI tract of a subject. The controlled release dosage form of the present invention may be designed to deliver the self-emulsifying nanosuspension of the present invention at a desired rate over a desired period of time. Typically a controlled release dosage form of the present invention will be designed to deliver the self-emulsifying nanosuspension of the present invention at a desired release rate over a period of time ranging from about 1 hour to about 24 hours. In a preferred embodiment, the controlled release dosage form of the present invention is designed to begin delivery of the self-emulsifying nanosuspension only after the dosage form has entered the lower GI tract of a subject. As they are used herein, the phrases “lower GI” or “lower GI tract” or “lower portions of the GI tract” indicate the distal small intestine and the colon.

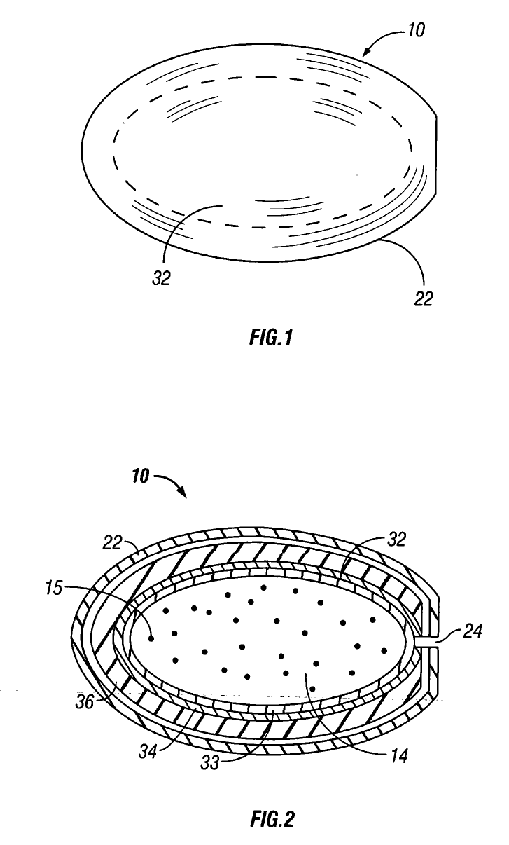

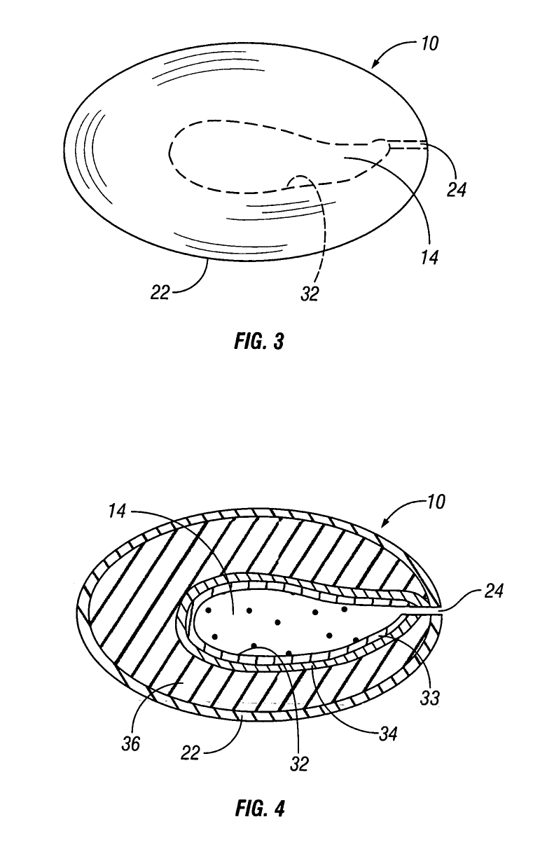

Though a controlled release dosage form may be designed to provide the controlled release of the self-emulsifying nanosuspension of the present invention using any release or delivery mechanism that provides for the release of self-emulsifying nanosuspension at a desired rate over a desired period of time, a controlled release dosage form of the present invention is preferably an osmotic dosage form. Osmotic dosage forms, such as those described in U.S. Pat. Nos. 6,419,952, 6,342,249, 6,183,466, 6,174,547, 5,614,578, 5,413,572, 5,324,280, and 4,627,850, which are assigned to ALZA Corporation and which are incorporated herein by reference, are desirable because the expandable osmotic material included in these dosage forms works to expel flowable drug formulations at a controlled rate in environments having relatively small amounts of aqueous media, such as the lower GI tract.

Where the controlled release dosage form of the present invention is an osmotic dosage form, the dosage form may be formed using a soft capsule or hard capsule as described in U.S. Pat. Nos. 6,419,952, 5,614,578, 5,413,572, and 5,324,280 and in U.S. patent application 60/343,001, and 60/343,005, the contents of which are incorporated herein by reference. FIGS. 1 through 14 illustrate a preferred embodiments of a controlled release dosage form according to the present invention formed using a soft gelatin capsule.

Where a soft gelatin capsule, or “soft-cap,” is used to form the controlled release dosage form 10 of the present invention, the dosage form 10 includes a soft-cap 32 containing a self-emulsifying nanosuspension 14. A barrier layer 34 is formed around the soft-cap 32, and a layer of expandable osmotic material 36, or “osmotic layer,” is formed around the barrier layer 34. A soft-cap controlled release dosage form 10 according to the present invention is provided with a semipermeable membrane 22, the semipermeable membrane 22 being formed over the osmotic layer 36. An exit orifice 24 is preferably formed through the semipermeable membrane 22, the osmotic layer 36, and the barrier layer 34 to facilitate delivery of the self-emulsifying nanosuspension 14 from the soft-cap controlled release dosage form 10.

The soft-cap 32 used to create a controlled release dosage form 10 of the present invention may be a conventional gelatin capsule, and may be formed in two sections or as a single unit capsule in its final manufacture. Preferably, due to the presence of the barrier layer 34, the wall 33 of the soft-cap 32 retains its integrity and gel-like characteristics, except where the wall 33 dissolves in the area exposed at the exit orifice 24. Generally maintaining the integrity of the wall 33 of the soft-cap 32 facilitates well-controlled delivery of the formulation 14. However, some dissolution of portions of the soft-cap 32 extending from the exit orifice 24 during delivery of the formulation 14 may be accommodated without significant impact on the release rate or release rate profile of the formulation 14.

Any suitable soft-cap may be used to form a controlled release dosage form according to the present invention. The soft-cap 32 may be manufactured in accordance with conventional methods as a single body unit comprising a standard capsule shape. Such a single-body soft-cap typically may be provided in sizes from 3 to 22 minims (1 minim being equal to 0.0616 ml) and in shapes of oval, oblong, or others. The soft cap 32 may be manufactured in accordance with conventional methods using, for example, a soft gelatin material or a hard gelatin material that softens during operation. The soft cap 32 may be manufactured in standard shapes and various standard sizes, conventionally designated as (000), (00), (0), (1), (2), (3), (4), and (5), with largest number corresponding to the smallest capsule size. However, whether the soft-cap 32 is manufactured using soft gelatin capsule or hard gelatin capsule that softens during operation, the soft-cap 32 may be formed in non-conventional shapes and sizes if required or desired for a particular application.

At least during operation, the wall 33 of the soft-cap 32 should be soft and deformable to achieve a desired release rate or release rate profile. The wall 33 of a soft-cap 32 used to create a controlled release dosage form 10 according to the present invention will typically have a thickness that is greater than the thickness of the wall 13 of a hard-cap 12 used to create a hard-cap controlled release dosage form 10. For example, soft-caps may have a wall thickness on the order of 10-40 mils, with about 20 mils being typical, whereas hard-caps may have a wall thickness on the order of 2-6 mils, with about 4 mils being typical. U.S. Pat. Nos. 5,324,280 and 6,419,952 and U.S. applications numbered 60/343,001, and 60/343,005, the contents of which have already been incorporated herein by reference, describe the manufacture of various soft-caps useful for the creation of controlled release dosage form according to the present invention.

The barrier layer 34 formed around the soft-cap 32 is deformable under the pressure exerted by the osmotic layer 36 and is preferably impermeable (or less permeable) to fluids or materials that may be present in the osmotic layer 36 and in the environment of use during delivery of the self-emulsifying nanosuspension 14. The barrier layer 34 is also preferably impermeable (or less permeable) to the formulation 14 of the present invention. However, a certain degree of permeability of the barrier layer 34 may be permitted if the release rate or release rate profile of the self-emulsifying nanosuspension 14 is not detrimentally affected. As it is deformable under forces applied by osmotic layer 36, the barrier layer 34 permits compression of the soft-cap 32 as the osmotic layer 36 expands. This compression, in turn, forces the self-emulsifying nanosuspension 14 from the exit orifice 24. Preferably, the barrier layer 34 is deformable to such an extent that the barrier layer 34 creates a seal between the osmotic layer 36 and the semipermeable layer 22 in the area where the exit orifice 24 is formed. In that manner, barrier layer 34 will deform or flow to a limited extent to seal the initially exposed areas of the osmotic layer 36 and the semipermeable membrane 22 when the exit orifice 24 is being formed. Materials and methods suitable for forming a barrier layer 34 included in a soft-cap controlled release dosage form 10 of the present invention are taught in U.S. patent application 60/343,001, and 60/343,005.

The osmotic layer 36 included in a soft-cap controlled release dosage form 10 according to the present invention includes a hydro-activated composition that expands in the presence of water, such as that present in gastric fluids. The osmotic layer 36 may be prepared using the materials and methods described in U.S. Pat. Nos. 5,324,280 and 6,419,952, and in U.S. patent application serial No. 60/392,775, the contents of each of which are herein incorporated by this reference. As the osmotic layer 36 imbibes and/or absorbs external fluid, the osmotic layer 36 expands and applies a pressure against the barrier layer 34 and the wall 33 of the gel-cap 32, thereby forcing the self-emulsifying nanosuspension 14 through the exit orifice 24.

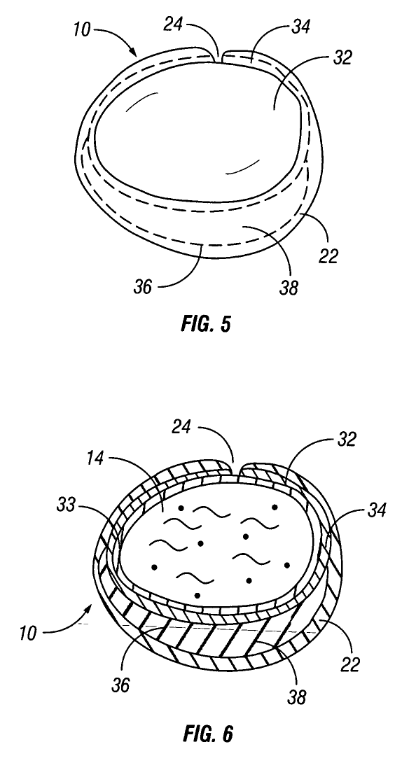

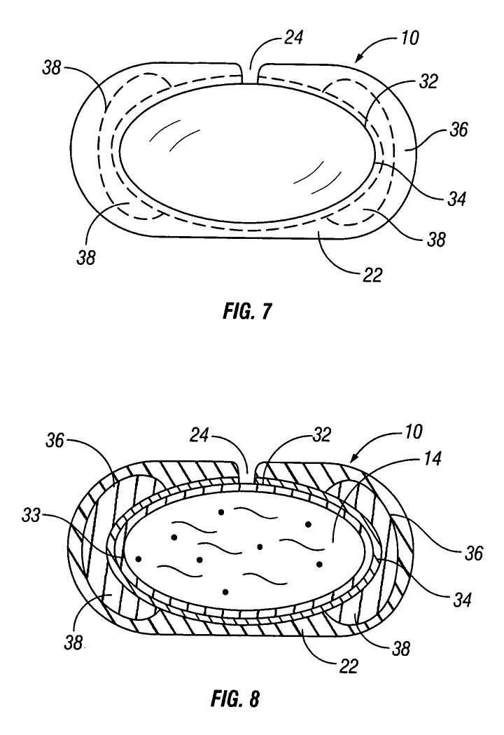

As shown in FIG. 1, FIG. 5-FIG. 8, and FIG. 10-FIG. 11, the osmotic layer 36 included in a soft-cap controlled release dosage form 10 of the present invention may be configured as desired to achieve a desired release rate or release rate profile and a desired delivery efficiency. For example, the osmotic layer 36 may be an unsymmetrical hydro-activated layer (shown in FIG. 5 and FIG. 6), having a thicker portion remote from the exit orifice 24. The presence of the unsymmetrical osmotic layer 36 functions to assure that the maximum dose of formulation 14 is delivered from the dosage form 10, as the thicker section of the osmotic layer 36 swells and moves towards the exit orifice 24. As is easily appreciated by reference to the figures, the osmotic layer 36 may be formed in one or more discrete sections 38 that do not entirely encompass the barrier layer 34 formed around the soft cap 32 (shown in FIG. 5-FIG. 8). As can be seen from FIG. 5 and FIG. 6, the osmotic layer 36 may be a single element 40 that is formed to fit the shape of the soft-cap 32 at the area of contact. Alternatively, the osmotic layer 36 may include two or more discrete sections 38 formed to fit the shape of the soft-cap 32 in the areas of contact (shown in FIG. 7 and FIG. 8).

The osmotic layer 36 may be fabricated as a tableted material using known materials and fabrication techniques. For example, the osmotic layer maybe fabricated conveniently by tableting to form an osmotic layer 36 of a desired shape and size. For example, the osmotic layer 36 may be tableted as a concave surface that is complementary to the external surface of the barrier layer 34 formed on the soft-cap 32. Appropriate tooling such as a convex punch in a conventional tableting press can provide the necessary complementary shape for the osmotic layer. Where formed by tableting, the osmotic layer 36 is granulated and compressed, rather than formed as a coating. Methods of forming an osmotic layer by tableting are described, for example, in U.S. Pat. Nos. 4,915,949, 5,126,142, 5,660,861, 5,633,011, 5,190,765, 5,252,338, 5,620,705, 4,931,285, 5,006,346, 5,024,842, and 5,160,743, the contents of which are incorporated herein by reference.

The semipermeable membrane 22 formed around the osmotic layer 36 is non-toxic and maintains its physical and chemical integrity during operation of the soft-cap controlled release dosage form 10. The semipermeable membrane 22 is permeable to the passage of water but is substantially impermeable to the passage of the active agent included in the self-emulsifying nanosuspension 14. The semipermeable membrane 22 is non-toxic to the intended subject and maintains its physical and chemical integrity during the operation of the dosage form 10. Further, adjusting the thickness or chemical make-up of the semipermeable membrane 22 can control the rate at which the expandable osmotic composition 36 included in the dosage form 10 expands. Therefore, the semipermeable membrane 22 coating a dosage form 10 of the present invention may be used to control the release rate or release rate profile achieved by the the dosage form 10.

The semipermeable membrane 22 included in a controlled release dosage form of the present invention may be formed using any material that is permeable to water, is substantially impermeable to the active agent, is pharmaceutically acceptable, and is compatible with the other components of the dosage form. Generally, the semipermeable membrane 22 will be formed using materials that include semipermeable polymers, semipermeable homopolymers, semipermeable copolymers, and semipermeable terpolymers. Semipermeable polymers are known in the art, as exemplified by U.S. Pat. No. 4,077,407, which is incorporated herein by reference, and they can be made by procedures described in Encyclopedia of Polymer Science and Technology, Vol. 3, pages 325 to 354, 1964, published by Interscience Publishers, Inc., New York. The semipermeable membrane 22 included in the dosage form 10 of the present invention may also include a flux regulating agent, such as a flux enhancing or a flux reducing agent, to assist in regulating the fluid permeability or flux through the semipermeable membrane 22. Additional references describing materials and methods suitable for fabricating the semipermeable membrane 22 included in the dosage form 10 of the present invention include, U.S. Pat. Nos. 6,174,547, 6,245,357, and 6,419,952 and U.S. patent application Ser. Nos. 08/075,084, 09/733,847, 60/343,001, 60/343,005, and 60/392,774, the contents which are incorporated herein by reference.

It is presently preferred that a soft-cap controlled-release dosage form 10 of the present invention include mechanism for sealing any portions of the osmotic layer 36 exposed at the exit orifice 24. Such a sealing mechanism prevents the osmotic layer 36 from leaching out of the system during delivery of formulation 14. In one embodiment, the exit orifice 24 is drilled and the exposed portion of the osmotic layer 36 is sealed by barrier layer 34, which, because of its rubbery, elastic-like characteristics, flows outwardly about the inner surface of exit orifice 24 during and/or after the formation of the exit orifice 24. In that manner, the barrier layer 34 effectively seals the area between the osmotic layer 34 and semipermeable layer 22. This can be seen most clearly in FIG. 4. In order to flow and seal, the barrier layer 34 should have a flowable, rubbery-like consistency at the temperature at which the system operation takes place. Materials, such as copolymers of ethyl acrylate and methyl methacrylate, especially Eudragit NE 30D supplied by RohmPharma, Darmstaat, Germany, are preferred. A soft-cap controlled release dosage form 10 having such a sealing mechanisms may be prepared by sequentially coating the soft-cap 32 with a barrier layer 34, an osmotic layer 36, and semipermeable layer 22 and then drilling the exit orifice 24 to complete the dosage form 10.

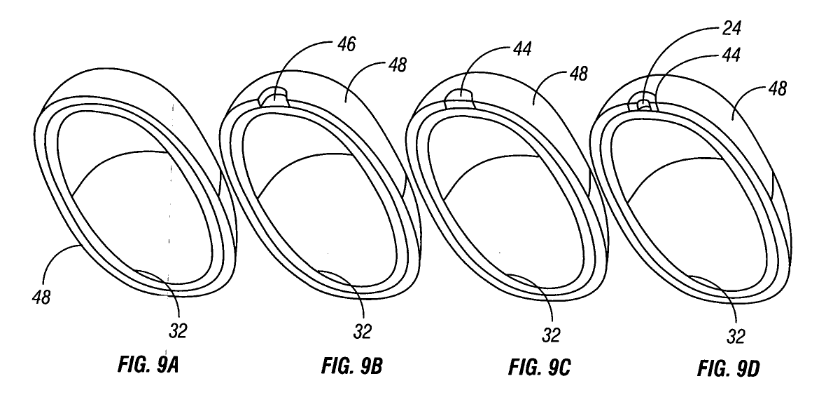

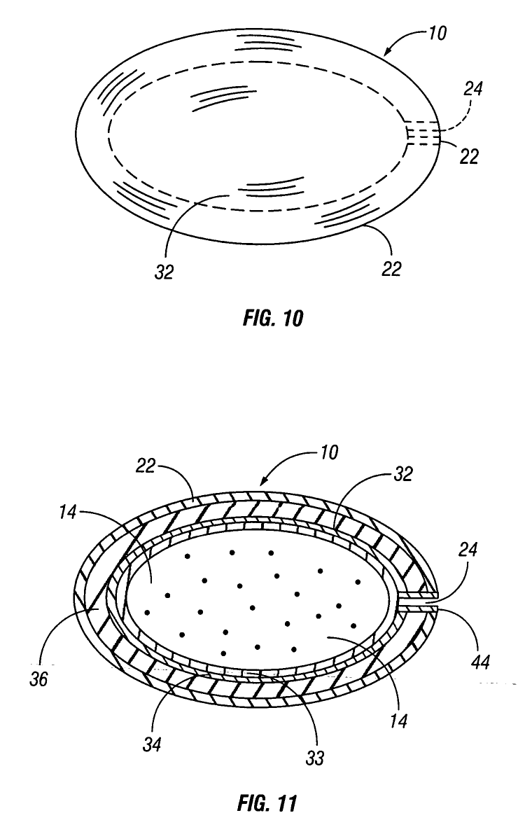

Alternatively a plug 44 may be used to form the desired sealing mechanism for the exposed portions of the osmotic layer 36. As is shown in FIG. 9A through FIG. 9D, a plug 44 may be formed by providing a hole 46 in the semipermeable membrane and the barrier layer (shown as a single composite membrane 48). The plug 44 is then formed by filling the hole 46 with, for example, a liquid polymer that can be cured by heat, radiation or the like (shown in FIG. 9C). Suitable polymers include polycarbonate bonding adhesives and the like, such as, for example, Loctite® 3201, Loctite® 3211, Loctite® 3321 and Loctite® 3301, sold by the Loctite Corporation, Hartford, Conn. The exit orifice 24 is drilled into plug to expose a portion of the soft-cap 32. A completed dosage form having a plug-type seal is illustrated in an overall view of FIG. 10 and in cross-section in FIG. 11.

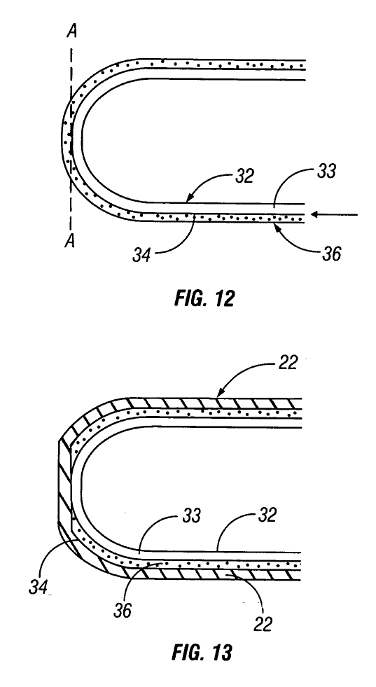

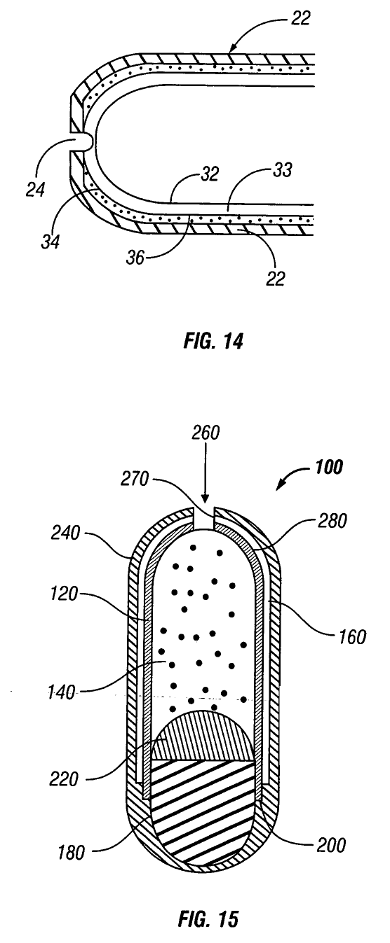

Still another manner of preparing a dosage form having a seal formed on the inner surface of the exit orifice is described with reference to FIG. 12-FIG. 14. In FIG. 12, a soft-cap 32 (only partially shown) has been coated with the barrier layer 34 and an osmotic layer 36. Prior to coating the semipermeable membrane 22, a section of the osmotic layer 36 extending down to, but not through, the barrier layer 34 is removed along line A-A. Then a semipermeable membrane 22 is coated onto the dosage form 10 to yield a precursor of the dosage form such as illustrated in FIG. 13. As can be seen from FIG. 13, the portion of gel-cap 32 where the exit orifice 24 is to be formed is covered by the semipermeable membrane 22 and the barrier layer 34, but not the osmotic layer 36. Consequently, when an exit orifice 24 is formed in that portion of the dosage form 10, as can be seen most clearly in FIG. 14, the barrier layer 34 forms a seal at the juncture of the semipermeable membrane 22 and expandable layer 20 such that fluids may pass to osmotic layer 36 only through the semipermeable membrane 22. Accordingly, osmotic layer 36 is not leached out of the dosage form 10 during operation. The sealing aspect of the soft-cap controlled release dosage form 10 of the present invention allows the rate of flow of fluids to the osmotic layer 36 to be carefully controlled by controlling the fluid flow characteristics of the semipermeable membrane 22.

In the embodiment shown in FIG. 5 and FIG. 6, the barrier layer 34 is first coated onto the gelatin capsule 12 and then the tableted, osmotic layer 36 is attached to the barrier-coated soft-cap with a biologically compatible adhesive. Suitable adhesives include, for example, starch paste, aqueous gelatin solution, aqueous gelatin/glycerin solution, acrylate-vinylacetate based adhesives such as Duro-Tak adhesives (National Starch and Chemical Company), aqueous solutions of water soluble hydrophilic polymers such as hydroxypropyl methyl cellulose, hydroxymethyl cellulose, hydroxyethyl cellulose, and the like. That intermediate dosage form is then coated with a semipermeable membrane. The exit orifice 24 is formed in the side or end of the soft-cap 32 opposite the osmotic layer 36. As the osmotic layer 36 imbibes fluid, it will swell. Since it is constrained by the semipermeable membrane 22, the osmotic layer 36 compresses the soft-cap 32 as the osmotic layer 36 expands, thereby expressing the formulation 14 from the interior of the soft-cap 32 into the environment of use.

As mentioned, the soft-cap controlled release dosage form 10 of the present invention may include an osmotic layer formed of a plurality of discrete sections. Any desired number of discrete sections may be used, but typically the number of discrete sections will range from 2 to 6. For example, two sections 38 may be fitted over the ends of the barrier-coated soft-cap 32 as illustrated in FIG. 12 and FIG. 13. FIG. 12 is a schematic of a soft-cap controlled release dosage form 10 with the various components of the dosage form indicated by dashed lines and the soft-cap 32 indicated by a solid line. FIG. 13 is a cross-sectional view of a completed soft-cap controlled release dosage form 10 having two, discrete expandable sections 38. Each expandable section 38 is conveniently formed by tableting from granules and is adhesively attached to the barrier-coated soft-cap 32, preferably on the ends of the soft-cap 32. Then a semipermeable layer 22 is coated on the intermediate structure and an exit orifice 24 is formed in a side of the dosage form between the expandable sections 38. As the expandable sections 38 expand, the formulation 14 will be expressed from the interior of the soft-cap 32 in a controlled manner to provide controlled-release delivery of the self-emulsifying nanosuspension 14.

The controlled release dosage form of the present invention may also be manufactured using a hard-cap, such as a capsule fabricated of hard gelatin or polymer materials. U.S. Pat. Nos. 6,174,547, 5,413,572 and 5,614,578 and U.S. patent application 60/392,774, which have already been incorporated herein by reference, teach exemplary controlled release dosage forms that may be used to deliver the self-emulsifying nanosuspension of the present invention and can serve as a controlled release dosage form of the present invention. A presently preferred hard-cap controlled release dosage form is illustrated in FIG. 15.

As can be seen by reference to FIG. 15, the preferred controlled release hard-cap dosage form 100 includes a capsule body 120 filled with a self-emulsifying nanosuspension 140. A water impermeable subcoat 160 may be provided on the outer surface of the capsule body 120, and an expandable osmotic composition 180 is positioned within a first end 20 of the capsule body 120. If desired, a barrier layer 220 may be positioned between the expandable osmotic composition 180 and the self-emulsifying nanosuspension 140. Where included, a barrier layer 220 works to prevent mixing of the self-emulsifying nanosuspension 140 with the expandable osmotic composition 180 and serves to ensure more complete delivery of the self-emulsifying nanosuspension 140 from the dosage form 100 as the expandable osmotic composition 180 expands during operation. As can be seen in FIG. 15, a semipermeable membrane 240 is formed over the water impermeable subcoat 16 and any exposed portions of the capsule body 120 and the expandable osmotic composition 180. To facilitate expulsion of the self-emulsifying nanosuspension 140, a dosage form 100 of the present invention also includes an exit orifice 260, which is preferably formed in an area near a second end 280 of the capsule body 120. As is shown in FIG. 15, the exit orifice 260 will generally be formed at a location opposite the expandable osmotic composition 180.

The capsule body 120 included in a preferred hard-cap controlled release dosage form 100 of the present invention is formed to contain a desired amount of self-emulsifying nanosuspension 140 and includes a first end 200 and a second end 280. The first end 200 of the capsule body 120 is open and is sized and shaped to accommodate the expandable osmotic composition 180. As can be seen in FIG. 15, the capsule body 120 of the dosage form 100 does not include a cap and does not encapsulate the expandable osmotic composition 180. In this manner, contact between the capsule body 120 and the expandable osmotic composition 180 prior to the operation of the dosage form 100 is reduced relative to previous dosage form designs. Such a design reduces capsule cracking due to water migration from the capsule material into the osmotic composition, and thereby reduces the likelihood that interaction between the expandable osmotic composition 180 and the capsule body 120 will affect the structural stability of the capsule body 120 either before or during operation of the dosage form 100. Though the capsule body 120 illustrated in FIG. 15 is formed in a generally oblong shape, the capsule body of a controlled release hard-cap dosage form 100 of the present invention is not so limited and may be sized and shaped as desired to contain a desired amount of liquid active agent formulation or to suit a particular drug delivery application.

To further reduce the problems associated with hydration sensitivity, the preferred embodiment of the controlled release hard-cap dosage form 100 of the present invention may include a capsule body 120 formed of a water-soluble polymer material. Relative to gelatin materials, water-soluble polymer materials are less susceptible to moisture loss and are markedly less sensitive to changes in moisture content than the gelatin materials typically used in capsule fabrication. Polymer materials that can be used to form the capsule body 120 include, for example, polysaccharide materials, such as hydroxypropylmethyl cellulose (HPMC), methylcellulose, hydroxyethyl cellulose (HEC), hydroxypropyl cellulose (HPC), poly(vinylalcohol-co-ethylene glycol) and other water soluble polymers suitable for dip-coating or extrusion processes for making capsule bodies. Though the capsule body 120 included in the preferred hard-cap controlled release dosage form 100 may be manufactured using a single polymer material, the capsule body 120 may also be formed using a mixture of more than one polymer material. Presently, HPMC capsules are preferably used to form a hard-cap capsule body 120 because they are commercially available and provide desirable performance characteristics. However, the capsule body 120 included in a hard-cap controlled release dosage form according to the present invention may be formed using a variety of materials and methods, with exemplary materials and methods being described in, for example, the U.S. Pat. Nos. 6,174,547, 5,413,572 and 5,614,578 and U.S. patent application 60/392,774, the contents of which are incorporated herein by reference.

Where included, the optional water impermeable subcoat 160 formed on the capsule body 120 of a hard-cap controlled release dosage form 100 of the present invention works to minimize or prevent the migration of water from an external environment, through the capsule body 120, and into the self-emulsifying nanosuspension 140. In order to be effective, the water impermeable subcoat 160 need not be perfectly impermeable to the passage of water. As it is used herein, the expression “water impermeable” refers to subcoats exhibiting a water flux of less than about 10-4 (mil·cm/atm-hr). Any material that provides a subcoat of sufficient water impermeability, is pharmaceutically acceptable, and is compatible with the other components of the dosage form may be used to form the water impermeable subcoat 160. However, latex materials, such as Surelease® latex materials available from Colorcon, Inc., Kollicoat® SR latex materials available from BASF, Eudragit® SR, and other polymethylacrylate latex materials, are presently preferred for forming the water impermeable subcoat 160.

A water impermeable subcoat 160 may be provided on the capsule body 120 using any suitable coating technique. For example, the capsule body 120 may be provided with a water impermeable subcoat 160 using a known dip coating process. A water impermeable subcoat 160 may also be formed over the capsule body 120 using a spray coating process. Where a spray coating process is used, however, the capsule body 120 is preferably provided with a removable cap before the spray coating is conducted. Providing the capsule body 120 with a removable cap prior to the spray coating process prevents coating of the interior surface of the capsule body 120 with the material forming the water impermeable subcoat 160. Once the spray coating process is complete, however, the cap should be readily removable to allow further processing of the coated capsule body 120. An exemplary spray coating process suitable for providing a capsule body 120 included in a hard-cap dosage controlled release dosage form according to the present invention with a water impermeable subcoat is described in U.S. patent application 60/392,774, the contents of which are incorporated herein by reference.

The expandable osmotic composition 180 included in a dosage form 100 of the present invention is formulated such that, the expandable osmotic composition 180 expands as it absorbs water from the environment of operation and exerts a force against the self-emulsifying nanosuspension 140, which causes the expulsion of the self-emulsifying nanosuspension 140 through the exit orifice 26. Any composition that exhibits such characteristics, is pharmaceutically acceptable, and is compatible with the other components of the dosage form of the present invention may be used to form the expandable osmotic composition 180. Exemplary materials and methods for forming an expandable osmotic composition 180 for use a controlled release hard-cap dosage form 100 of the present invention are detailed in U.S. Pat. Nos. 6,174,547 6,245,357, and 6,419,952 and in U.S. patent application Ser. Nos. 09/733,847, 60/343,001, and 60/343,005, and 60/392,774, the contents of which are incorporated herein by reference.

As can also be appreciated by reference to FIG. 15, the expandable osmotic composition 180 of the a controlled release hard-cap 100 according to the present invention is preferably tableted in a bi-layer tablet 30 including a barrier layer 220. The barrier layer 220 works to minimize or prevent the mixing of the self-emulsifying nanosuspension 140 with the expandable osmotic composition 180 before and during operation of the dosage form 100. By minimizing or preventing mixing of the self-emulsifying nanosuspension 140 with the expandable osmotic composition 180, the barrier layer 220 serves to reduce the amount of residual active agent remaining within the dosage form 100 after the expandable osmotic composition 180 has ceased expansion or has filled the interior of the dosage form 100. The barrier layer 220 also serves to increase the uniformity with which the driving power of the expandable osmotic composition 180 is transferred to the self-emulsifying nanosuspension 140 included in the dosage form 100. A barrier layer 220 included in the preferred hard-cap controlled release dosage form 100 may be formed using the materials and methods described in U.S. Pat. No. 6,419 application Ser. Nos. 08/075,084, 60/343,001, 60/343,005, and 60/392,774.

The semipermeable membrane 240 included on the a controlled release hard-cap dosage form 100 of the present invention is permeable to the passage of water but is substantially impermeable to the passage of the active agent included in the self-emulsifying nanosuspension 140. The semipermeable membrane 240 is non-toxic to the intended subject and maintains its physical and chemical integrity during the operation of the dosage form 100. Further, adjusting the thickness or chemical make-up of the semipermeable membrane 240 can control the rate at which the expandable osmotic composition 180 of included in the dosage form 100 of the present invention expands. Therefore, the semipermeable membrane 240 coating a dosage form 100 of the present invention may be used to control the release rate or release rate profile achieved by the preferred controlled release hard-cap dosage form 100. The semipermeable membrane 240 provided in a hard-cap controlled release dosage form of the present invention may be provided using the materials and methods already described in relation to the preferred-soft cap controlled release dosage form illustrated in FIGS. 1 through 14.

The exit orifice 260 included in a hard-cap controlled release dosage form 100 of the present invention may be embodied by one of various different structures suitable for allowing the release of the self-emulsifying nanosuspension 140. As illustrated in FIG. 15, the exit orifice 26 is generally formed at or near the second end 280 of the capsule body 120 and may include an aperture 270 formed through the semipermeable membrane 240 and the water impermeable subcoat 160. The aperture 270 of the exit orifice 260 illustrated in FIG. 15 exposes a portion of the capsule body 120 but preferably does not penetrate the capsule body 120. Upon administration of the dosage form 100 to an environment of operation, water present in the environment of operation weakens and dissolves the portion of the capsule body 120 exposed by the aperture 270, allowing the self-emulsifying nanosuspension 140 contained within the capsule body 120 to be expelled. Though the exit orifice 260 illustrated in FIG. 15 is only one of various different exit orifices that may be provided in a hard-cap controlled release dosage form according to the present invention, the exit orifice 260 shown in FIG. 15 is advantageous, as it does not require penetration of the capsule body 120 before the dosage form 100 is administered. Such a design works to prevent leaking of the self-emulsifying nanosuspension 140 from the dosage form 100 before the dosage form 100 is administered. Moreover, the aperture 270 shown in FIG. 15 is simply formed using known mechanical or laser drilling techniques. Nevertheless, a controlled release hard-cap dosage form 100 of the present invention is not limited to the exit orifice 260 illustrated in FIG. 15. Descriptions of various embodiments of exit orifices that may be used in a hard-cap controlled release dosage form of the present invention are disclosed, for example, in those patents and patent applications already incorporated herein by reference, as well as in U.S. Pat. Nos. 3,845,770, 3,916,899, and 4,200,098, the contents of which are herein incorporated by reference.

The hard-cap and soft-cap controlled release dosage forms prepared in accordance with the present invention may be constructed as desired to provide controlled release of the formulation of the present invention at a desired release rate or release rate profile over a desired period of time. Preferably, the controlled release dosage forms of the present invention are designed to provide controlled release of the formulation of the present invention over a prolonged period of time. As used herein, the phrase “prolonged period of time” indicates a period of time of two or more hours. Typically for human and veterinary pharmaceutical applications, a desired prolonged period of time may be from 2 hours to 24 hours, more often 4 hours to 12 hours or 6 hours to 10 hours. For many applications it may be preferable to provide dosage forms that only need to be administered once a day.

In a particularly preferred embodiment, the controlled release dosage form of the present invention is designed to begin release of a self-emulsifying nanosuspension contained therein only after the dosage form has entered the lower GI tract of a subject. In one such embodiment, the controlled release dosage form of the present invention is provided with and enteric overcoat that works to prevent operation of the dosage form until the dosage form has entered the lower GI tract of a subject. Enteric coatings are known in the art and are designed to remain intact until exposed to an aqueous environment having a predetermined pH. Therefore, a controlled release dosage form can be according to the present invention can be provided with an enteric coating that remains intact in the upper GI tract of a subject but dissolves the in the lower GI tract due to the change in pH that occurs as the dosage form travels from the upper portions of the GI tract to the lower potions of the GI tract. Exemplary enteric coatings are discussed at, for example, Remington 's Pharmaceutical Sciences, (1965), 13th ed., pages 604-605, Mack Publishing Co., Easton, Pa.; Polymers for Controlled Drug Delivery, Chapter 3, CRC Press, 1991; Eudragit® Coatings Rohm Pharma, (1985); and U.S. Pat. No. 4,627,851. If desired, the thickness and chemical constituents of an enteric coating formed on a dosage form of the present invention may be selected to target release of the formulation of the present invention within a specific region of the lower GI tract.

Of course, a controlled release dosage form of the present invention designed to begin release of the self-emulsifying nanosuspension after passage through the upper GI is not limited to a controlled release dosage form having an enteric coating. For instance, the semipermeable membrane, osmotic composition, and self-emulsifying nanosuspension may be formulated and designed such that the controlled release dosage form does not begin delivery of the self-emulsifying nanosuspension for a period of time that is sufficient to generally ensure passage of the dosage form through the upper GI tract and into the lower GI tract of the subject. Alternatively, a controlled release dosage form according to the present invention may be designed to begin delivery the self-emulsifying nanosuspension of the present invention in the lower GI tract of a subject by providing a controlled release dosage form with an outer coating that erodes over a desired period of time after administration, with the erosion of the coating being substantially independent of environmental pH.

### EXAMPLE 1

Megestrol acetate is a synthetic progestin indicated for palliative treatment of various cancers, such as breast, endometrial and prostate cancers. The water solubility of megestrol acetate is about 2 μg/ml at 37° C. Due to its poor water solubility megestrol acetate exhibits a low oral bioavailability.

A first self-emulsifying nanosuspension according to the present invention containing megestrol acetate was prepared. The megestrol acetate used in this and all other examples was supplied by Diosynth Corporation of the Netherlands. The first nanosuspension was prepared by dispersing megestrol acetate nanoparticle in capric acid and Cremophor EL. The nanoparticles were prepared by wet milling (using Dyno milling equipment) followed by freeze-drying. Pluronic F108 was used as a coating agent in the wet milling process. The mean particle size of the nanoparticles was 0.3 μm as measured by Horiba LA-910 laser scattering particle size analyzer. The megestrol acetate was dispersed within the capric acid and Cremophor EL using a sonicator, with the resulting self-emulsifying nanosuspension including 3.8 wt % megestrol acetate nanoparticle, 1.4 wt % Pluronic F108, 47.4 wt % capric acid, and 47.4 wt % Cremophor EL.

A first batch of hard-cap controlled release dosage forms according to the present invention was then manufactured using the first self-emulsifying formulation. The first dosage forms were prepared using a clear, size-0 hard-caps. The first dosage forms incorporated a bi-layer osmotic composition and were coated with a rate controlling semipermeable membrane. An exit orifice was provided in the first dosage forms using a mechanical drill with drilling depth control.

To prepare the bi-layer osmotic composition used in the dosage forms, an osmotic granulation of was prepared using a Glatt fluid bed granulator (FBG). The osmotic granulation included NaCl, NaCMC, HPMC, HPC, Mg stearate and red ferric oxide. The NaCl was sized/screened using a Quardo mill having a 21-mesh screen and the speed set on maximum. The sized NaCl, NaCMC, HPMC, and red ferric oxide were blended in a granulator bowl in the following weight percentages: 58.75% NaCMC, 30% sized/screened NaCl, 5.0% HPMC E-5 and 1.0% red ferric oxide. In a separate container, a granulating solution was prepared by dissolving 5.0 wt % HPC EF in purified water. The osmotic granulation was then prepared by spraying the granulation solution onto the fluidized powders until all of the solution was applied and the powders were granular. A final osmotic granulation was completed by blending 0.25 wt % Mg stearate with the prepared granules.

The barrier layer included in the bi-layer osmotic composition included in the first hard-cap controlled release dosage forms was formed using Kollidon SR. The final osmotic granulation was used to prepare a bi-layer osmotic composition by compressing an amount of the final osmotic granulation and an amount of Kollidone SR into a bi-layer tablet using Carver tableting press. Two hundred and seventy mg of the final osmotic granulation was added to a 0.70 cm punch (lower punch: modified ball, upper punch: modified) and tamped. 80 mg of Kollidone SR was then added to the punch and the osmotic granulation and Kollidone SR were compressed under a force of about 1 metric ton to form a tableted bi-layer osmotic composition.

To load the self-emulsifying nanosuspension into the capsules used to prepare the first hard-caps, the capsules were separated into two segments (a body and a cap). The self-emulsifying nanosuspension was then loaded into the body of each capsule using standard filling techniques. Each capsule was provided with 526 mg of the self-emulsifying nanosuspension. The megestrol acetate dose of the resulting hard-cap controlled release dosage form was, therefore, about 20 mg. After the capsule bodies were filled, pre-coating assemblies were formed by positioning a bi-layer osmotic composition in each filled capsule body.

The pre-coating assemblies were then coated with a semipermeable membrane. The semipermeable membrane was provided over the pre-coating assemblies included, by weight, 70% cellulose acetate 398-10 and 30% Pluronic F-68. To form the semipermeable membrane a coating composition was first formed by dissolving appropriate amounts of cellulose acetate 398-10 and Pluronic F-68 in acetone to form a coating solution having a solid content of 4% by weight. The pre-coating assemblies were then sprayed with the coating solution in a 12″ Freud Hi-coater until each was provided with a semipermeable membrane weighing about 131 mg.

After membrane coating, the first hard-cap controlled release dosage forms were completed by drying the coated sub-assemblies and providing each of the dried and coated sub-assemblies with an exit orifice. The coated sub-assemblies were dried in a Blue oven at 30° C. overnight, and each of the dried sub-assemblies was then provided with an exit orifice measuring about 0.5 mm in diameter. The exit orifices were provided in each dosage form by drilling the drug-layer side using a mechanical drill with drilling depth control.

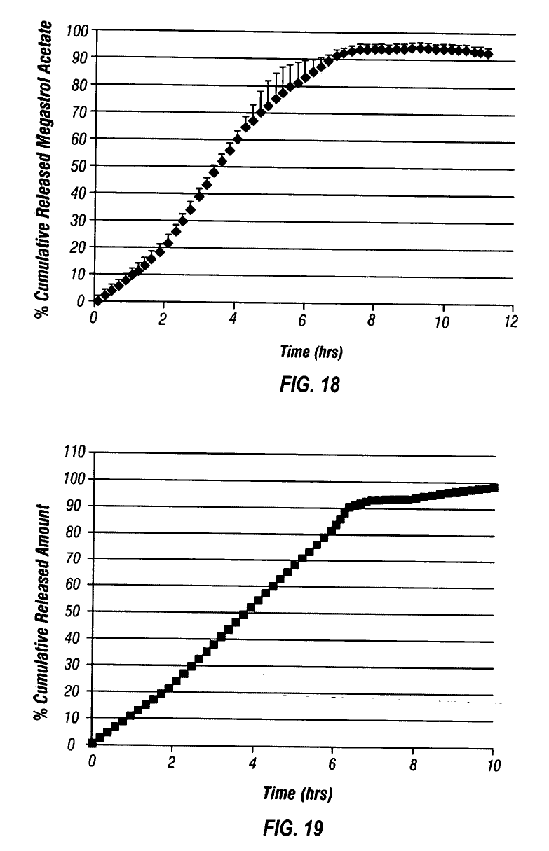

The release rate profile of the first hard-cap controlled release dosage forms was measured using a USP II paddle method in 2%, by weight, aqueous solution of Pluronic F108 (pH 6.8). As shown in FIG. 18, 90% of the megestrol acetate contained in the dosage forms was released at a substantially constant rate over about 7 hrs.

### EXAMPLE 2

A second self-emulsifying nanosuspension according to the present invention was prepared using the materials described in EXAMPLE 1. However, the second self-emulsifying nanosuspension was prepared to include relatively more megestrol acetate nanoparticle. Using the methods described in EXAMPLE 1, the second self-emulsifying nanosuspension was prepared to include 16 wt % megestrol acetate nanoparticle, 4.2 wt % Pluronic F108, 39.9 wt % of capric acid and 39.9 wt % Cremophor EL.

A second batch of hard-cap controlled release dosage forms according to the present invention was prepared using the second self-emulsifying nanosuspension. The capsules used to fabricate the second hard-cap controlled release dosage form were #2 hard-caps. The osmotic composition of the second hard-cap controlled release dosage forms was manufactured using the same osmotic granulation and barrier layer material as described in EXAMPLE 1, but the weights of the materials included in the bi-layer osmotic composition varied from those described in EXAMPLE 1. To provide the bi-layer osmotic composition included in the second hard-cap controlled release dosage form, 180 mg of the osmotic granulation and 70 mg of the barrier-layer material (Kollidon SR) were compressed to the bi-layer tablets using 227′ concaved, flat tooling. After the 180 mg of osmotic granulation and 70 mg of Kollidon SR were tableted to form a bi-layer osmotic composition, an additional amount of Kollidon SR was added to the barrier layer by compressing 130 mg of Kollidon SR over the compressed barrier material already present using the same tooling. The additional amount of Kollidon SR served to fill empty space present in the #2 capsule body.

The bodies of the capsules used to form the second hard-cap controlled release dosage forms were separated and 125 mg of the second self-emulsifying was loaded into each capsule body. Because the self-emulsifying nanosuspension loaded into the second hard-cap controlled release dosage form included 16% megestrol acetate by weight, each of the completed second hard-cap controlled release dosage forms contained about a 20 mg dose of megestrol acetate. Once the capsule bodies were filled with the desired amount of self-emuslfying nanosuspension, the bi-layer osmotic compositions were positioned in the capsule bodies to form pre-coating assemblies.

The rate-controlling membrane included over the pre-coating assemblies of the second hard-cap controlled release dosage form was composed of 90% cellulose acetate 398-10 and 10% Pluronic F-68. The coating solution used to produce the semipermeable membrane of the second hard-cap controlled release dosage form was prepared by dissolving appropriate amounts of cellulose acetate 398-10 and Pluronic F-68 in acetone to provide a coating solution including 4% solids, by weight. Each of the coating sub-assemblies of the second hard-cap controlled release dosage form was then coated in a in a 12″ Freud Hi-coater until each sub-assembly was provided with a semipermeable membrane weighing about 47 mg. To complete the second hard-cap controlled release dosage forms, the coated sub-assemblies were then dried and provided an exit orifice as described in EXAMPLE 1

The release rate profile provided by the second hard-cap controlled release dosage forms was then evaluated according to the process outlined in EXAMPLE 1. As can be seen by reference to FIG. 19, 90% of the megestrol acetate contained in the dosage forms was released at a substantially constant rate over about 7 hrs.

### EXAMPLE 3

The solubility of raw megestrol acetate and nanoparticulate megestrol acetate in AIF was evaluated in the presence of various concentrations of an exemplary self-emulsifying carrier. The exemplary self-emulsifying carrier included a blend of saturated fatty acid and surfactant (capric acid/Cremphor EL: 50/50, by wt), and the solubility of megestrol acetate was measured at 37° C. Different samples of AIF media were prepared with various concentrations (0.0, 0.1, 0.2, 0.5, 1.0%, w/w) of the self-emulsifying carrier. The megestrol acetate was added in excess into each AIF sample, and shaken overnight at 37° C. After shaking, each AIF sample was centrifuged and the supernatant of each AIF sample was assayed using a UV spectrometer at 290 μm.

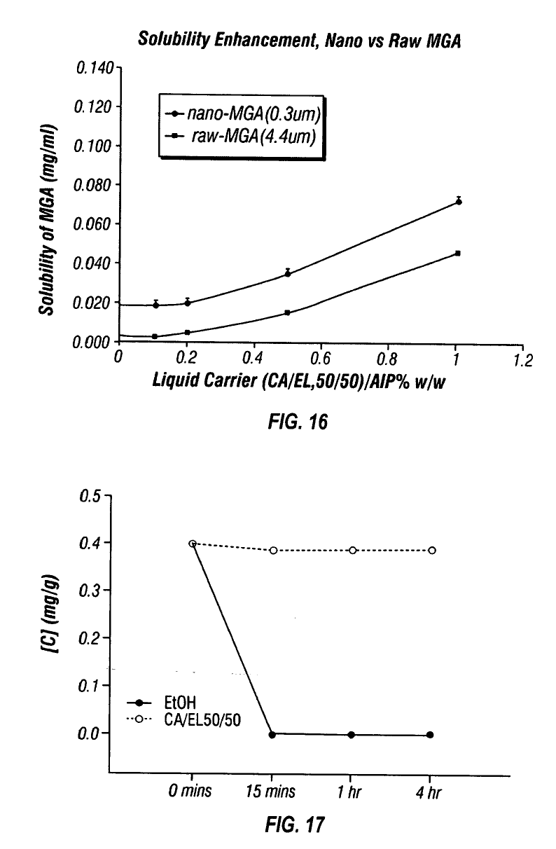

The results of the evaluation are provided in FIG. 16. As can be seen by reference to FIG. 16, the solubility of the nanoparticulate megestrol acetate was greater than the solubility of the raw megestrol acetate and the solubility of megestrol acetate increased and the concentration of self-emulsifying carrier increased.

### EXAMPLE 4

The stability of megestrol acetate solubilized in an emulsion formed by an exemplary self-emulsifying carrier was evaluated. The self-emulsifying carrier included 50 wt % capric acid and 50 wt % Cremophor EL 50/50. Solutions of megestrol acetate in the self-emulsifying carrier and in ethanol were prepared, and the megestrol acetate concentration for each solution was 20 mg/g. After the solutions were prepared, 0.2 g of each solution was added into 10 ml of AIF. The mixtures were shaken in a water bath at 37° C., and mixture samples were taken at time intervals of 15 mins, 60 mins, and 4 hrs. These samples were measured for megestrol acetate concentration after being filtered through 0.2 μm filter.

FIG. 17 illustrates the results of the evaluation. As shown in FIG. 17, no precipitation of megestrol acetate was noticed in the AIF containing megestrol acetate solubilized in the self-emulsifying carrier. In contrast, the megestrol acetate contained within the ethanol solution precipitated out within the first 15 minutes after introduction of the ethanol solution into the AIF.

### EXAMPLE 5

A five-arm PK study was conducted to evaluate the bioavailability of megestrol acetate provided by several different dosage forms. The study included administering various dosage forms to three fasted mongrel dogs. The dosage forms administered in the study included controlled release dosage forms manufactured according to EXAMPLE 1 (“4% nanosuspension hard-cap”) and EXAMPLE 2 (“16% nanosuspension hard-cap”), commercially available 20 mg Megace® tablets, hard-cap controlled release dosage forms containing a self-emulsifying solution of megestrol acetate (“controlled release SES dosage forms”), and immediate release hard-caps containing a self-emulsifying solution of megestrol acetate (“IR SES dosage forms”). The formulations delivered by the different dosage forms are described in Table 2.

The controlled release SES dosage forms were prepared using the methods and materials described in EXAMPLE 1, except that the drug formulation contained in the controlled release SES dosage forms was a self-emulsifying solution, not a suspension. The self-emulsifying solution loaded in the controlled release SES dosage forms included, by weight, 1.77% megestrol acetate and 0.83% Pluronic F108 dissolved in 48.7% Cremophor EL and 48.7% capric acid. The compounds included in the self-emulsifying solution were mixed using a mechanical agitator. Each of the controlled release SES dosage forms were filled with 565 mg of the self-emulsifying solution, with each of the controlled release SES dosage forms providing a 10 mg dose of megestrol acetate. As is shown in FIG. 2, the controlled release SES dosage forms delivered 90% of the megestrol acetate in about 7 hours after administration.

The IR SES dosage forms were prepared simply by filling a #0 hard with the same self-emulsifying solution used in the controlled release SES dosage forms. Each of the IR SES dosage forms was loaded with 565 mg of self emulsifying solution and, therefore, provided a 10 mg dose of megestrol acetate.

The dosage forms were dosed to the dogs in a fasted state using oral gavage. The same group of three dogs was used throughout the study, with each of the three dogs being dosed with each of the different dosage forms. In each arm of the study, the dogs were given a 20 mg dose of megestrol acetate. In order to achieve a 20 mg dose, each dog was administered two controlled release SES dosage forms and two IR SES dosage forms, as each of these dosage forms provided only a 10 mg dose of megestrol acetate. Plasma samples were taken from each dog at 0, 0.5, 1, 2, 4, 6, 8, and 10 hours after dosing each of the dosage forms, with additional plasma samples being taken from each dog at 12 hours and 24 hours after administration of the three controlled release dosage forms. The plasma concentration of megestrol acetate in each sample was evaluated using an LC/MS method with a minimum detection limit of 1 ng/ml. The LC/MS conditions are provided in Table 3.

AUCinf was calculated by adding AUCt and AUCt-inf., where AUCt was estimated by trapezoidal integration to the last sampling point (t) and AUCt-inf was estimated by integration from t to infinity.

AUC_{t-inf}=Cₜ/k

Where Ct, K and t are, respectively, the drug concentration of the plasma sample at the last sampling point t, the apparent elimination rate constant, and the last sampling time. K was estimated by linear regression of log plasma concentration at the terminal phase versus time. The average relative BA % was calculated as follows.

$$BA \% = 100 \times [ {\sum }_{i = 1}^{3} {( \frac{{AUC}_{inf}^{SEF}}{{AUC}_{inf}^{tablet}} )}_{i} ] / 3$$

Where AUC_{inf}^{SEF} and AUC_{inf}^{tablet} are AUC of SEF dosage forms and AUC of Megace tablet, respectively.

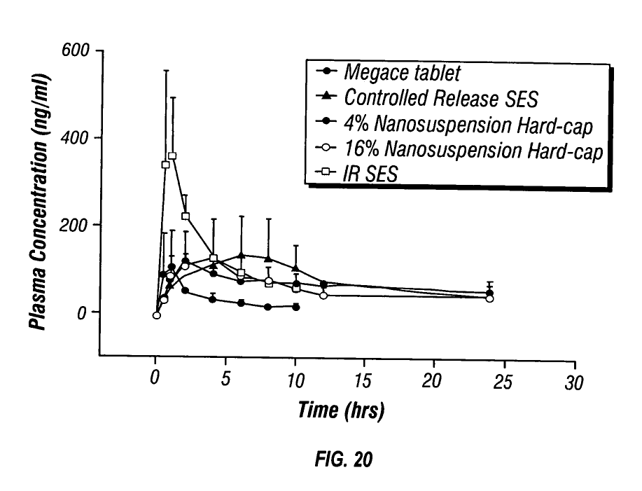

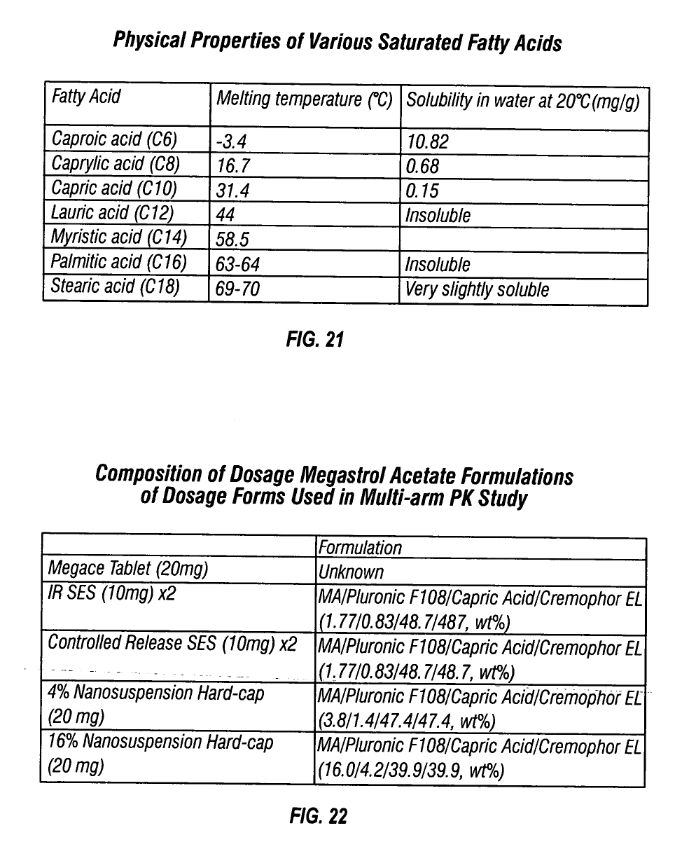

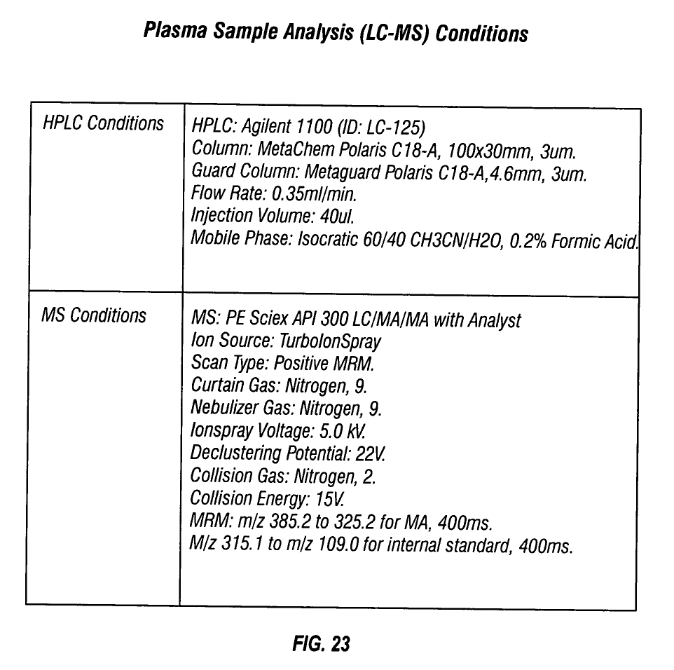

The results of the PK study are shown in FIG. 20. As can be seen in this figure, both the 4% nanosuspension hard-cap and the 16% nanosuspension hard cap provided a more than four-fold increase in bioavailability of megestrol acetate compared to the Megace® 20 mg tablet. Moreover, while providing significantly increased drug loading, the 4% nanosuspension hard cap and 16% nanosuspension hard cap provided megestrol acetate bioavailabilities that were comparable to those provided by the controlled release SES dosage form.

We claim:

1. A drug formulation comprising:
a hydrophobic drug in nanoparticulate form;
an oil phase comprising a saturated fatty acid; and
a surfactant, wherein the surfactant and saturated fatty acid are selected and combined
such that the drug formulation automatically forms a stable emulsion upon introduction to an aqueous media.

2. The drug formulation of claim 1, wherein the hydrophobic drug comprises a drug classified as a Class II drug under the Biopharmaceutics Classification System.

3. The drug formulation of claim 1, wherein the hydrophobic drug exhibits a dose/solubility volume of more than 250 ml.

4. The drug formulation of claim 1, wherein the hydrophobic drug comprises particles of hydrophobic drug that are smaller than about 1 μm in all dimensions.

5. The drug formulation of claim 1, wherein the hydrophobic drug comprises particles of hydrophobic drug that are smaller than about 0.5 μm in all dimensions.

6. The drug formulation of claim 1, wherein the hydrophobic drug comprises particles of hydrophobic drug that are smaller than about 0.2 μm in all dimensions.

7. The drug formulation of claim 1, wherein the hydrophobic drug comprises a drug selected from the group consisting of antibacterial agents, antiviral agents, anti-fungal agents, antacids, anti-inflammatory substances, coronary vasodilators, cerebral vasodilators, psychotropics, antineoplastics, stimulants, antihistamines, laxatives, decongestants, vitamins, anti-diarrheal preparations, anti-anginal agents, vasodilators, anti-arrythmics, anti-hypertensives, vasoconstrictors, anti-migraine drugs, antineoplastic drugs, anticoagulants, anti-thrombotic drugs, analgesics, anti-pyretics, neuromuscular agents, agents acting on the central nervous system, hyperglycemic agents, hypoglycemic agents, thyroid and anti-thyroid preparations, diuretics, anti-spasmodics, uterine relaxants, mineral and nutritional additives, anti-obesity agents, anabolic agents, ani-asthmatics, expectorants, cough suppressants, mucolytics, and anti-uricemic drugs

8. The drug formulation of claim 1, wherein the hydrophobic drug comprises a drug selected from the group consisting of poorly soluble proteins, polypeptides, peptides, proteomimetic and peptidomimetic materials.

9. The drug formulation of claim 1, wherein the fatty acid comprises a saturated C8 through a C12 fatty acid.

10. The drug formulation of claim 1, wherein the fatty acid comprises a saturated C10 fatty acid.

11. The drug formulation of claim 1, wherein the fatty acid comprises capric acid.

12. The drug formulation of claim 1, wherein the fatty acid comprises a blend of fatty acids selected from saturated C8 through C12 fatty acids.

13. The drug formulation of claim 1, wherein the fatty acid comprises 10 wt % to 80 wt % of the drug formulation.

14. The drug formulation of claim 1, wherein the fatty acid comprises 35 wt % to 45 wt % of the drug formulation.

15. The drug formulation of claim 1, wherein hydrophobic drug comprises a drug that exhibits a solubility in the oil phase that is at least 10 times greater than the solubility of the drug in water.

16. The drug formulation of claim 1, wherein hydrophobic drug comprises a drug that exhibits a solubility in the oil phase that is at least 100 times greater than the solubility of the drug in water.

17. The drug formulation of claim 1, wherein hydrophobic drug comprises a drug that exhibits a solubility in the oil phase that is at least 500 times greater than the solubility of the drug in water.

18. The drug formulation of claim 1, wherein the hydrophobic drug comprises from 2 wt % to 50 wt % of the drug formulation.

19. The drug formulation of claim 1, wherein the drug formulation comprises a first amount of hydrophobic drug dissolved within the oil phase and a second amount of hydrophobic drug suspended as a nanoparticulate material, with the first amount of hydrophobic drug and the second amount of hydrophobic drug accounting for 2 wt % to 50 wt % of the drug formulation.

20. The drug formulation of claim 1, wherein the hydrophobic drug comprises from 10 wt % to about 30 wt % of the drug formulation.

21. The drug formulation of claim 1, wherein the drug formulation comprises a first amount of hydrophobic drug dissolved within the oil phase and a second amount of hydrophobic drug suspended as a nanoparticulate material, with the first amount of hydrophobic drug and the second amount of hydrophobic drug accounting for 10 wt % to 30 wt % of the drug formulation.

22. The drug formulation of claim 1, wherein the surfactant comprises a non-ionic surfactant.

23. The drug formulation of claim 1, wherein the surfactant comprises a non-ionic surfactant and accounts for 5 wt % to 90 wt % of the drug formulation.

24. The drug formulation of claim 1, wherein the surfactant comprises a non-ionic surfactant and accounts for 25 wt % to 45 wt % of the drug formulation.

25. The drug formulation of claim 1, wherein the surfactant is selected from the group consisting of polyoxyethylene products of hydrogenated vegetable oils, polyethoxylated castor oils, polyethoxylated hydrogenated castor oils, polyoxyehtylene-sorbitan-fatty acid esters, polyoxyethylene castor oil derivatives, and pluronic surfactants.

26. The drug formulation of claim 1, wherein the surfacant is selected from the group consisting of polyoxyethylenated castor oil comprising 9 moles of ethylene oxide, polyoxyethylenated castor oil comprising 15 moles of ethylene oxide, polyoxyethylenated castor oil comprising 25 moles of ethylene oxide, polyoxyethylenated castor oil comprising 35 moles of ethylene oxide, polyoxyethylene castor oil comprising 40 moles of ethylene oxide, polyoxylenated castor oil comprising 52 moles of ethylene oxide, polyoxyethylenated sorbitan monopalmitate comprising 20 moles of ethylene oxide, polyoxyethylenated sorbitan monostearate comprising 20 moles of ethylene oxide, polyoxyethylenated sorbitan monostearate comprising 4 moles of ethylene oxide, polyoxyethylenated sorbitan tristearate comprising 20 moles of ethylene oxide, polyoxyethylenated sorbitan monostearate comprising 20 moles of ethylene oxide, polyoxyethylenated sorbitan trioleate comprising 20 moles of ethylene oxide, polyoxyethylenated stearic acid comprising 8 moles of ethylene oxide, polyoxyethylene lauryl ether, polyoxyethylenated stearic acid comprising 40 moles of ethylene oxide, polyoxyethylenated stearic acid comprising 50 moles of ethylene oxide, polyoxyethylenated stearyl alcohol comprising 2 moles of ethylene oxide, and polyoxyethylenated oleyl alcohol comprising 2 moles of ethylene oxide.

27. The drug formulation of claim 1, wherein the surfactant is selected from the group consisting of NIKKOL HCO-50®, NIKKOL HCO-35®, NIKKOL HCO-40®, NIKKOL HCO-60®, CREMAPHORE®, CREMAPHORE RH40®, CREMAPHORE RH60®, CREMAPHORE RH410®, CREMAPHORE RH455®, and CREMAPHORE EL®, TWEEN 20®, TWEEN 21®, TWEEN 40®, TWEEN 60®, TWEEN 80®, TWEEN 81®, Pluronic F68, Pluronic F108, and Pluronic F127.

28. The drug formulation of claim 1, wherein the surfactant is included in the drug formulation in an amount sufficient to cause the drug formulation to automatically form a stable microemulsion upon introduction to an aqueous media.

29. A drug formulation formed of a nanosuspension of a hydrophobic drug, the drug formulation comprising:
a hydrophobic drug material in nanoparticulate form;
an oil phase comprising a saturated C8 through a C12 fatty acid, wherein the hydrophobic drug material exhibits a solubility in the oil phase that is at least 10 times greater than the solubility of the hydrophobic drug material in water; and
a non-ionic surfactant, wherein the non-ionic surfactant and oil phase are selected and combined such that the drug formulation automatically forms a stable emulsion upon introduction to an aqueous media.

30. The drug formulation of claim 29, wherein hydrophobic drug comprises a drug classified as a Class II drug under the Biopharmaceutics Classification System, the drug exhibiting a dose/solubility volume of more than 250 ml.

31. The drug formulation of claim 30, wherein the hydrophobic drug is selected from the group consisting of hydrophobic drug materials exhibiting an average particle size that is smaller than about 1 μm in all dimensions, hydrophobic drug materials exhibiting an average particle size that is smaller than about 0.5 μm in all dimensions, and hydrophobic drug materials exhibiting an average particle size that is smaller than about 0.2 μm in all dimensions.

32. The drug formulation of claim 1, wherein the fatty acid comprises 35 wt % to 45 wt % of the drug formulation, and the non-ionic surfactant comprises 25 wt % to 45 wt % of the drug formulation.

33. The drug formulation of claim 32, wherein the wherein the drug formulation comprises a first amount of hydrophobic drug dissolved within the oil phase and a second amount of hydrophobic drug suspended as a nanoparticulate material, with the first amount of hydrophobic drug and the second amount of hydrophobic drug accounting for 10 wt % to 40 wt % of the drug formulation.

34. The drug formulation of claim 29, wherein the surfactant is included in the drug formulation in an amount sufficient to cause the drug formulation to automatically form a stable microemulsion upon introduction to an aqueous media.

35. A drug formulation formed of a nanosuspension of a hydrophobic drug, the drug formulation comprising:
a hydrophobic drug material in nanoparticulate form, wherein the hydrophobic drug material comprises a drug exhibiting a dose/solubility volume of more than 250 ml and is selected from the group consisting of hydrophobic drug materials exhibiting an average particle size that is smaller than about 1 μm in all dimensions, hydrophobic drug materials exhibiting an average particle size that is smaller than about 0.5 μm in all dimensions, and hydrophobic drug materials exhibiting an average particle size that is smaller than about 0.2 μm in all dimensions;
an oil phase comprising a saturated C8 through a C12 fatty acid, wherein the hydrophobic drug material exhibits a solubility in the oil phase that is at least 100 times greater than the solubility of the hydrophobic drug material in water; and
a non-ionic surfactant, wherein the non-ionic surfactant and oil phase are selected and combined such that the drug formulation automatically forms a stable microemulsion upon introduction to an aqueous media.

36. The drug formulation of claim 35, wherein the fatty acid comprises 35 wt % to 45 wt % of the drug formulation, and the non-ionic surfactant comprises 25 wt % to 45 wt % of the drug formulation.

37. The drug formulation of claim 36, wherein the drug formulation comprises a first amount of hydrophobic drug dissolved within the oil phase and a second amount of hydrophobic drug suspended as a nanoparticulate material, with the first amount of hydrophobic drug and the second amount of hydrophobic drug accounting for 10 wt % to 40 wt % of the drug formulation.

38. The drug formulation of claim 1, wherein the hydrophobic drug, the oil phase, and the surfactant are selected and combined such that the drug formulation provides at least a four-fold increase in the bioavailability of the hydrophobic drug when delivered from a controlled release dosage form relative to a tableted, immediate release formulation of the drug.

39. The drug formulation of claim 29, wherein the hydrophobic drug, the oil phase, and the surfactant are selected and combined such that the drug formulation provides at least a four-fold increase in the bioavailability of the hydrophobic drug when delivered from a controlled release dosage form relative to a tableted, immediate release formulation of the drug.

40. The drug formulation of claim 35, wherein the hydrophobic drug, the oil phase, and the surfactant are selected and combined such that the drug formulation provides at least a four-fold increase in the bioavailability of the hydrophobic drug when delivered from a controlled release dosage form relative to a tableted, immediate release formulation of the drug.
